(19) United States
(12) Patent Application Publication
(10) Pub. No.: US 2022/0096393 A1
(43) Pub. Date: Mar. 31, 2022

(54) TRANSDERMAL SYSTEM WITH OVERPLASTER AND RING SYSTEM

(71) Applicant: LTS Lohmann Therapie-Systeme AG, Andernach (DE)

(72) Inventors: Florian HAMMES, Andernach (DE); Anja TOMELERI, Neuwied (DE); Tobias KLEUDGEN, Ettringen (DE)

(73) Assignee: LTS Lohmann Therapie-Systeme AG, Andernach (DE)

(21) Appl. No.: 17/295,101

(22) PCT Filed: Nov. 20, 2019
(86) PCT No.: PCT/EP2019/081912

§ 371 Date: May 19, 2021

(30) Foreign Application Priority Data
Nov. 29, 2018 (DE) 10 2018 220 589.2

(51) Int. Cl. A61K 9/70

(52) U.S. Cl. CPC A61K 9/7084

(57) ABSTRACT

The invention relates to a system containing: a transdermal therapeutic system (TTS); a cylindrical separating layer having an outer periphery and an inner periphery; and a backing layer, which is applied to the cylindrical separating layer and protrudes beyond the cylindrical separating layer over the entire periphery. The invention further relates to a system for improving the adhesion of a TTS, to the use of the system, and to a kit containing the system.

The present invention relates to a transdermal system for use in clinical and preclinical studies, to the use of a cylindrical release layer in these studies, and to a method for the determination of pharmacological and physical data. The present invention further relates to a system for improving the adhesion of a transdermal therapeutic system (TTS), to the use of this system, and to a kit that includes this system.

For the development of new transdermal therapeutic systems (TTS), it is necessary to test these systems in preclinical and clinical studies. In preclinical and clinical studies, problems arise if the applied active-substance-containing transdermal therapeutic systems adhere poorly or only inadequately to the skin. The applied transdermal therapeutic systems are complete transdermal therapeutic systems, but in some cases the adhesive elements have still not been optimized. In addition, the initial studies are usually carried out on minipigs. The nature of the skin of minipigs is very similar to the nature of human skin, but the adhesive force of a TTS is adversely affected by the bristles of the minipigs. Furthermore, the considerable mechanical stresses in studies with minipigs pose a challenge for the adhesive force of a TTS. Therefore, the transdermal therapeutic systems are not optimized with respect to the adhesion to the skin of the minipigs. With a prolonged application time and/or difficult application sites and/or mechanical stresses, the TTS may detach completely from the skin and fall off in the worst case.

In the case of commercially available transdermal therapeutic systems too, there are systems which, for example on account of being worn for a long period of time, are subject to an increased risk of premature detachment of the TTS from human skin.

In. order to prevent premature detachment, the use of an overplaster is described for systems which do not have sufficient adhesion, for example in DE 102009022915 A1. Such an overplaster leads, on the one hand, to better adhesion of the TTS to the skin and, on the other hand, protects the TTS from mechanical stress.

When using such overplasters, the problem often arises that the transdermal therapeutic system (TTS) and the overplaster can no longer be released from each other after completion of the application. In preclinical and clinical studies, it is necessary for the TTS to be evaluated analytically after completion of the application. The overplaster affects this analytical evaluation or even makes it impossible. Furthermore, there is a risk of the active substance migrating from the TTS over the edges of the backing cover of the TTS and into the adhesive layer of the overplaster.

The object of the present invention was to make available a system in which there is no premature detachment of the TTS and in which there is no migration of the active substance over the edges of the backing cover of the TTS into the overplaster.

This object is achieved by a system comprising:
(i) a transdermal therapeutic system (TTS) with a side facing toward the skin and a side facing away from the skin;
(ii) a cylindrical release layer with an outer circumference, an inner circumference, a front face and a bottom face, wherein the cylindrical release layer is applied to the side of the TTS facing away from the skin, and the outer circumference of the cylindrical release layer protrudes fully circumferentially over the TTS, and the TTS protrudes fully circumferentially over the inner circumference of the cylindrical release layer;
(iii) an overplaster which is applied to the cylindrical release layer and protrudes fully circumferentially over the latter.

According to the invention, the outer circumference of the cylindrical release layer protrudes fully circumferentially over the TTS, and the TTS protrudes fully circumferentially over the inner circumference of the cylindrical release layer. This means that the TTS and the cylindrical release layer overlap. The TTS is a flat system which has an outer circumference larger than the inner circumference of the cylindrical release layer, but smaller than the outer circumference of the cylindrical release layer. Since the outer circumference of the cylindrical release layer is larger than the outer circumference of the TTS, the cylindrical release layer lies over the edges of the backing cover of the TTS. As a result, the edges of the TTS do not come into contact with the overplaster.

To ensure that there is no migration of the active substance over the edges of the backing cover of the TTS into the overplaster, it is preferable that the cylindrical release layer is impermeable to active substance.

There are no specific limitations as regards the size of the inner circumference of the cylindrical release layer, provided that the inner circumference of the cylindrical release layer is smaller than the outer circumference of the TTS, such that the TTS protrudes fully circumferentially over the inner circumference of the cylindrical release layer. The inner circumference of the release layer encloses an area whose area content is preferably from 0.1 to 30.0 cm², particularly preferably from 1.0 to 15.0 cm², very particularly preferably from 5.0 to 10.0 cm².

Since the cylindrical release layer is a layer which has an inner circumference, the overplaster can come into contact with the TTS within the area which is formed by the inner circumference of the cylindrical release layer. When the overplaster and the TTS come into contact over the area formed by the inner circumference of the cylindrical release layer, the TTS adheres to the overplaster. This makes it possible to better fix the TTS, and the latter does not slip beneath the overplaster.

Furthermore, the cylindrical release layer preferably has a thickness of 5 to 20 μm, particularly preferably of 8 to 17 μm, very particularly preferably of 10 to 15 μm.

There are no specific limitations as regards the region in which the TTS protrudes over the inner circumference of the cylindrical release layer. The TTS preferably protrudes fully circumferentially over the inner circumference of the cylindrical release layer by at least 1 mm, particularly preferably by at least 3 mm, very particularly preferably by at least 5 mm.

It is further preferred that the outer circumference of the cylindrical release layer protrudes fully circumferentially over the TTS by at least 1 mm, particularly preferably by at least 3 mm, very particularly preferably by at least 5 mm.

There are no specific limitations as regards the shape of the cylindrical release layer, provided that the cylindrical release layer has an outer circumference, an inner circumference, a front face and a bottom face. The outer circumference and the inner circumference of the cylindrical release layer have, independently of each other, a circular, oval or polygonal shape, If the release layer has a polygonal shape, it is preferable that the corners are rounded.

The outer circumference and the inner circumference can have an identical or different shape. The outer circumference and the inner circumference preferably have an identical shape. The outer circumference and the inner circumference of the cylindrical release layer particularly preferably have a round or oval shape. It is preferable that the outer circumference of the release layer corresponds to the outer shape of the TTS used, and that the outer shape of the release layer protrudes over the outer shape of the TTS.

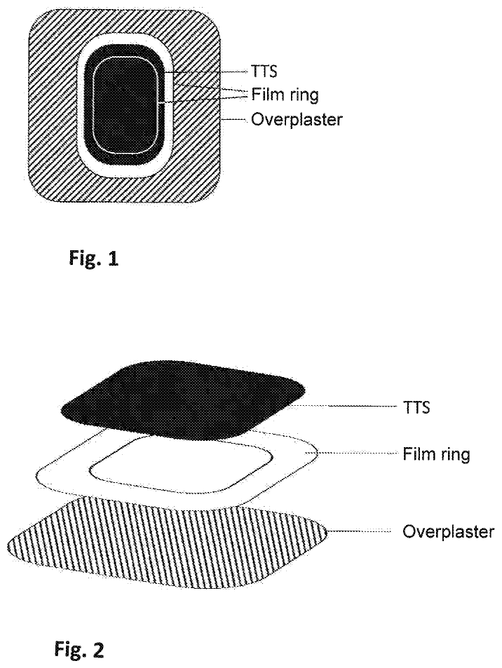

FIG. 1 shows a view of the system according to the invention from. below, that is to say from the skin side.

FIG. 2 shows a side view of the system according to the invention.

The system according to the invention comprises a TTS (1), a film ring (2) and an overplaster (3), the film ring representing the cylindrical release layer.

There are no specific limitations as regards the material from which the cylindrical release layer is constructed. The cylindrical release layer can be designed to be adhesive or non-adhesive. The cylindrical release layer is preferably made of a material selected from the group comprising polyethylene terephthalate, plasticized vinyl acetate-vinyl chloride copolymers, nylon, ethylene-vinyl acetate copolymers, plasticized polyvinyl chloride, polyurethane, polyvinylidene chloride, polypropylene, polyethylene or polyamide,

The overplaster is preferably made of a textile fabric. The fabric can be elastic in one or two directions. To ensure that the overplaster can be securely fixed to the skin and to the actual TTS, it is equipped with a pressure-sensitive adhesive layer.

Suitable base polymers for a pressure-sensitive adhesive layer are described, for example, in Tan, Pfister, PSTT vol. 2, no. 2 February 1999, pages 60-69. Suitable base polymers are, for example, polyacrylates, poly(meth)acrylates, polyacrylic acid, cellulose derivatives, in particular methyl and ethyl celluloses, polyisobutylene, ethylene-vinyl acetate copolymers, natural and synthetic rubbers such as styrene diene copolymers, styrene-butadiene block copolymers, styrene-isoprene block copolymers, acrylonitrile-butadiene rubber, butyl rubber or neoprene rubber, and also hot-melt adhesives. Those based on silicone can also he used as pressure-sensitive adhesives. Suitable mixtures of the polymers mentioned or hybrid adhesives from acrylate monomers and silicone monomers can also be used to advantage.

According to the invention, the pressure-sensitive adhesive layer with which the overplaster is provided contains, as base polymers, polymers preferably selected from the group comprising polyacrylates, poly(meth)acrylates, poiyacrylic acid, cellulose derivatives, polyisobutylene, ethylene-vinyl acetate copolymers, natural and synthetic rubbers such as styrene-diene copolymers, styrene-butadiene block copolymers, styrene-isoprene block copolymers, acrylonitrile-butadiene rubber, butyl rubber or neoprene rubber, and also hot-melt adhesives and silicone-based pressure-sensitive adhesives.

The TTS used in the system according to the invention is a complete TTS which comprises
(a) an active substance carrier or an active substance reservoir and
(b) a backing layer.

The backing layer of a TTS must be impermeable to the active substance contained in the TTS, in order to prevent the active substance from undesirably escaping from the side of the TTS facing away from the skin. For this purpose, special plastic films are used, e.g. metallized plastic films. Aluminized films of polyethylene terephthalate are preferably used. The advantage of using a metallized film is that it is impermeable to almost all pharmaceutical active substances and to light, and this, specifically in the case of light-sensitive active substances, offers the advantage of reliable light protection.

Particularly suitable materials for the active-substance-impermeable backing layer of the TTS are polyesters, which are distinguished by particular strength, for example polyethylene terephthalate and polybutylene terephthalate, but also almost any other skin-compatible plastics, such as polyvinyl chloride, polyurethane, polyvinylidene chloride, ethylene-vinyl acetate copolymers, polyvinyl acetate, vinyl acetate-vinyl chloride copolymers, nylon, polyethylene, polypropylene, polyurethanes, polyamide, cellulose derivatives and many others. Preference is given to polyethylene terephthalate, plasticized vinyl acetate-vinyl chloride copolymers, nylon, ethylene-vinyl acetate copolymers, plasticized polyvinyl chloride, polyurethane, polyvinylidene chloride, polypropylene, polyethylene and polyamide.

In the system according to the invention, the cylindrical release layer is applied to the backing layer of the TTS.

If the system according to the invention is intended to be evaluated analytically after completion of the application, the cylindrical release layer is preferably not adhesive. Such a system according to the invention is characterized in that the TTS, the cylindrical release layer and the overplaster are separable from one another. This simplifies the analytical evaluation after the application, since the analytical data are not distorted by the presence of the overplaster.

The present invention further relates to the use of a non-adhesive, cylindrical release layer with an outer circumference, an inner circumference, a front face and a bottom face, for the determination of pharmacological and physical data in clinical and preclinical studies.

The present invention further relates to a method for the determination of pharmacological and physical data, comprising:
(a) making available a system according to the invention, comprising:
(a1) applying a non-adhesive, cylindrical release layer with an outer circumference, an inner circumference, a front face and a bottom face to a TTS with a side facing toward the skin and a side facing away from the skin, such that the non-adhesive, cylindrical release layer is applied to the side of the TTS facing away from the skin, and the outer circumference of the non-adhesive, cylindrical release layer protrudes fully circumferentially over the TTS, and the TTS protrudes fully circumferentially over the inner circumference of the non-adhesive, cylindrical release layer;
(a2) applying an overplaster to the non-adhesive, cylindrical release layer, such that the overplaster protrudes fully circumferentially over the non-adhesive, cylindrical release layer;
(b) applying the system obtained from (a) to skin;
(c) collecting pharmacological and physical data.

The present invention further relates to a system for improving the adhesion of a TTS, comprising:
(ii) a cylindrical release layer with an outer circumference, an inner circumference, a front face and a bottom face;
(iii) an overplaster which is applied to the cylindrical release layer and protrudes fully circumferentially over the latter.

The cylindrical release layer (ii) and the overplaster (iii) are configured as described above.

The present invention also relates to the use of a system comprising:
(ii) a cylindrical release layer with an outer circumference, an inner circumference, a front face and a bottom face;
(iii) an overplaster which is applied to the cylindrical release layer and protrudes fully circumferentially over the latter;
   for improving the adhesion of a TTS and/or for preventing the active substance from migrating over the edges of the TTS.

Such a system is used together with a commercially available TTS, wherein the cylindrical release layer is applied to the side of the TTS facing away from the skin, and the outer circumference of the cylindrical release layer protrudes fully circumferentially over the TTS, and the TTS protrudes fully circumferentially over the inner circumference of the cylindrical release layer.

There are no specific limitations as regards the active substance used in the TTS, provided that the active substance is suitable for administration by means of a TTS. An example of such an active substance is buprenorphine. Examples of commercially available buprenorphine-containing TTS are Transtec®, Norspan® and Butrans®.

As has already been stated, the overplaster comes into contact with the TTS within the area formed by the inner circumference of the cylindrical release layer, and the TTS adheres to the overplaster. This makes it possible to fix a commercially available TTS and thus improve its adhesion to the skin, in such a system, there is also no migration of the active substance over the edges of the TTS, since the outer circumference of the cylindrical release layer protrudes fully circumferentially over the TTS.

The present invention further relates to a kit comprising:

a transdermal therapeutic system (TTS) and a system according to the invention for improving the adhesion of a TTS comprising:
(ii) a cylindrical release layer with an outer circumference, an inner circumference, a front face and a bottom face;
(iii) an overplaster which is applied to the cylindrical release layer and protrudes fully circumferentially over the latter.

1. A system comprising:
a transdermal therapeutic system (TTS) with a side facing toward skin and a side facing away from the skin;
(ii) a cylindrical release layer with an outer circumference, an inner circumference, a front face and a bottom face,
wherein the cylindrical release layer is applied to the side of the TTS facing away from the skin, and the outer circumference of the cylindrical release layer protrudes fully circumferentially over the TTS, and the TTS protrudes fully circumferentially over the inner circumference of the cylindrical release layer; and
(iii) an overplaster which is applied to the cylindrical release layer and protrudes fully circumferentially over the cylindrical release layer,
wherein the cylindrical release layer is impermeable to active substance, and the overplaster is equipped with a pressure-sensitive adhesive layer for fixing to the skin.

2. (canceled)

3. The system as claimed in claim 1, wherein the cylindrical release layer encloses an area whose area content is from 0.1 to 30.0 cm², and the cylindrical release layer has a thickness of 5 to 20 μm.

4. The system as claimed in claim 1, wherein the TTS protrudes fully circumferentially over the inner circumference of the cylindrical release layer by at least 1 mm, and the outer circumference of the cylindrical release layer protrudes fully circumferentially over the TTS by at least 1 mm, and the outer circumference and the inner circumference of the cylindrical release layer have, independently of each other, a circular, oval or polygonal shape.

5. The system as claimed in claim 1, wherein the cylindrical release layer is made of a material selected from polyethylene terephthalate, plasticized vinyl acetate-vinyl chloride copolymers, nylon, ethylene-vinyl acetate copolymers, plasticized polyvinyl chloride, polyurethane, polyvinylidene chloride, polypropylene, polyethylene or polyamide.

6. (canceled)

7. The system as claimed in claim 1, wherein the pressure-sensitive adhesive layer contains, as base polymers, polymers selected from the group comprising polyacrylates, poly(meth) acrylates, polyacrylic acid, cellulose derivatives, polyisobutylene, ethylene-vinyl acetate copolymers, natural rubbers, synthetic rubbers, hot-melt adhesives and pressure-sensitive adhesives based on silicone.

8. The system as claimed in claim 1, wherein the TTS comprises
(a) an active substance carrier or an active substance reservoir and
(b) a backing layer.

9. The system as claimed in claim 8, wherein the cylindrical release layer is applied to the backing layer of the TTS.

10. The system as claimed in claim 1, wherein the cylindrical release layer is not adhesive, and the TTS, the cylindrical release layer and the overplaster are separable from one another.

11. Clinical and preclinical studies comprising pharmacological and physical data based upon systems comprising a non-adhesive, cylindrical release layer with an outer circumference, an inner circumference, a front face and a bottom face.

12. A method for the determination of pharmacological and physical data, comprising:
(a) providing a system as claimed in claim 1, wherein the cylindrical release layer is non-adhesive;
(a1) applying the non-adhesive, cylindrical release layer with an outer circumference, an inner circumference, a front face and a bottom face to the TTS, with the TTS comprising a side facing toward skin and a side facing away from the skin, the non-adhesive, cylindrical release layer applied to the side of the TTS facing away from the skin, and the outer circumference of the non-adhesive, cylindrical release layer protruding fully circumferentially over the TTS, and the TTS protruding fully circumferentially over the inner circumference of the non-adhesive, cylindrical release layer;
(a2) applying an overplaster to the non-adhesive, cylindrical release layer, the overplaster protruding fully circumferentially over the non-adhesive, cylindrical release layer;
(b) applying the overplastered system obtained from (a2) to skin; and
(c) collecting pharmacological and physical data.

13. A system for improving the adhesion of a TTS comprising:
(ii) a cylindrical release layer with an outer circumference, an inner circumference, a front face and a bottom face; and
(iii) an overplaster applied to the cylindrical release layer which protrudes fully circumferentially over the cylindrical release layer.

14. A kit comprising:
a transdermal therapeutic system and
the system as claimed in claim 13.

15. A method for improving adhesion of a TTS and/or for preventing migration of an active substance over edges of the TTS comprising applying a system as claimed in claim 13 to the TTS.

16. The system as claimed in claim 3, wherein the cylindrical release layer encloses an area whose area content is from 1.0 to 15.0 cm², and the cylindrical release layer has a thickness of 8 to 17 μm.

17. The system as claimed in claim 3, wherein the cylindrical release layer encloses an area whose area content is from 5.0 to 10.0 cm², and the cylindrical release layer has a thickness of 10 to 15 μm.

18. The system as claimed in claim 4, wherein the TTS protrudes fully circumferentially over the inner circumference of the cylindrical release layer by at least 3 mm, and the outer circumference of the cylindrical release layer protrudes fully circumferentially over the TTS by at least 3 mm.

19. The system as claimed in claim 4, wherein the TTS protrudes fully circumferentially over the inner circumference of the cylindrical release layer by at least 5 mm, and the outer circumference of the cylindrical release layer protrudes fully circumferentially over the TTS by at least 5 mm.

20. The system as claimed in claim 7, wherein the natural and synthetic rubbers are selected from styrene-diene copolymers, styrene-butadiene block copolymers, styrene-isoprene block copolymers, acrylonitrile butadiene rubber, butyl rubber or neoprene rubber.
